(19) United States
(12) Patent Application Publication
(10) Pub. No.: US 2026/0083357 A1
(43) Pub. Date: Mar. 26, 2026

(54) Multi-Head Convolutional Network for Average Glucose Prediction

(71) Applicant: Google LLC, Mountain View, CA (US)

(72) Inventors: Dongeek Shin, San Jose, CA (US); William James Biederman, Menlo Park, CA (US)

(21) Appl. No.: 19/111,146

(22) PCT Filed: Sep. 2, 2022
(86) PCT No.: PCT/US2022/042467

§ 371 Date: Mar. 12, 2025

(51) Int. Cl. A61B 5/145; A61B 5/00; G06N 3/0464

(52) U.S. Cl. CPC A61B 5/14532; A61B 5/7264; A61B 5/746; G06N 3/0464

(57) ABSTRACT

Systems and methods for determining a glucose value for a user are disclosed herein. The method includes receiving a plurality of data inputs associated with biometric data of the user, the plurality of data inputs including at least one data input representative of a past estimated glucose value of the user and processing the plurality of data inputs with a multi-headed temporal convolutional neural network to generate a blood glucose value for the user. The method also includes providing a notification to the user based at least in part on the blood glucose value.

### FIELD

The present disclosure relates to determining an average glucose value for a user. In particular, aspects of the present disclosure can include a multi-head temporal convolutional neural network (“MH-TCNN”) that can analyze past average blood glucose levels and other relevant biometric data to predict an estimated average glucose value for the user.

### BACKGROUND

People living with diabetes may be required to wear continuous glucose monitoring (“CGM”) devices, which require that a user insert a needle into their bloodstream in order to monitor the average glucose levels in their blood. CGM devices can provide notifications to diabetic users when their average blood glucose levels become hyperglycemic (too high) or hypoglycemic (too low) and about trends relating to average blood glucose levels. However, if this device malfunctions, otherwise stops working, or if the user takes the device off, the user will not have a consistent way to monitor blood glucose levels. Therefore, a model for estimating a blood glucose level of the user is desired.

### SUMMARY

Aspects and advantages of embodiments of the present disclosure will be set forth in part in the following description, or can be learned from the description, or can be learned through practice of the embodiments.

One aspect of the present disclosure provides a method for determining a glucose value for a user. The method includes receiving a plurality of data inputs associated with biometric data of the user, the plurality of data inputs including at least one data input representative of a past estimated glucose value of the user and processing the plurality of data inputs with a multi-headed temporal convolutional neural network to generate a blood glucose value for the user. The method also includes providing a notification to the user based at least in part on the blood glucose value.

Another aspect of the present disclosure provides a computing device. The computing device includes one or more processors and a non-transitory, computer-readable medium comprising instructions that, when executed by the one or more processors, cause the one or more processors to perform a process. The process includes receiving a plurality of data inputs associated with biometric data of a user, the plurality of data inputs including at least one data input representative of a past estimated glucose value of the user and processing the plurality of data inputs with a multi-headed temporal convolutional neural network to generate a blood glucose value for the user. The process also includes providing a notification to the user based at least in part on the blood glucose value.

A further aspect of the present disclosure provides a non-transitory, computer-readable medium comprising instructions that, when executed by one or more processors, cause the one or more processors to perform a process. The process includes receiving a plurality of data inputs associated with biometric data of a user, the plurality of data inputs including at least one data input representative of a past estimated glucose value of the user and processing the plurality of data inputs with a multi-headed temporal convolutional neural network to generate a blood glucose value for the user. The process also includes providing a notification to the user based at least in part on the blood glucose value.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 illustrates a multi-head convolutional neural network for predicting an estimated glucose average according to one aspect of the present disclosure.

FIG. 2 illustrates a multi-head convolutional neural network for predicting an estimated glucose average according to one aspect of the present disclosure.

FIG. 3 illustrates buffer standardization for convolution layers in a multi-head convolutional neural network according to one aspect of the present disclosure.

FIG. 4 illustrates lifting non-temporal data inputs for a convolutional neural network according to one aspect of the present disclosure.

FIG. 5 illustrates patterns noticed in convolutional layers for various data inputs according to one aspect of the present disclosure.

FIG. 6 illustrates a method for predicting an estimated glucose value according to one aspect of the present disclosure.

FIG. 7 illustrates a wearable computing device according to one aspect of the present disclosure.

FIG. 8 illustrates a computing device communicating with a server according to one aspect of the present disclosure.

### DETAILED DESCRIPTION

Reference now will be made in detail to embodiments of the present disclosure, one or more examples of which are illustrated in the drawings. Each example is provided by way of explanation of the present disclosure, not limitation of the present disclosure. In fact, it will be apparent to those skilled in the art that various modifications and variations can be made in the present disclosure without departing from the scope or spirit of the disclosure.

For instance, features illustrated or described as part of one embodiment can be used with another embodiment to yield a still further embodiment. Thus, it is intended that the present invention covers such modifications and variations as come within the scope of the appended claims and their equivalents.

Aspects of the present disclosure include a multi-head temporal convolutional neural network (“MH-TCNN”) that can analyze past estimated glucose values, e.g., past average blood glucose levels and other relevant biometric data to generate or determine a blood glucose value of the user, e.g., predict an estimated average glucose value for the user. For example, the MH-TCNN future estimated glucose averages (“eGA”) data can be trained using eGA data associated with the specific user or average users with diabetes as well as supplementary physiological or biometric data, such as heart rate, to boost the estimation prediction confidence.

This is accomplished using the MH-TCNN by assigning each data input to a separate “head,” or convolution layer, that is used to learn underlying patterns of how various data inputs affect an output glucose level. The convolutional layers of a MH-TCNN may operate independently from one another. The output of each convolution layer is then provided to a final concatenated layer, which learns the underlying patterns of the outputs from each convolution layer to predict an estimated glucose value of the user's blood without the need for a present glucose value reading.

The MH-TCNN can be trained for each particular user and can iteratively learn from the user while in use. For example, the MH-TCNN can be operated on a computing device, such as a smart watch, that includes one or more biometric sensors that can gather biometric data about the user. In another example, the MH-TCNN can operate on a computing device that can receive biometric data, such as a smartphone, from various devices that include biometric sensors, such as smart watches, CGM devices, and the like. In other embodiments, the MH-TCNN can operate on a server that is in communicative contact with the computing device.

The advantage of using the MH-TCNN with supplementary data over normal convolutional neural networks results in a more accurate prediction, such as a much smaller mean-squared error (“MSE”) from the loss function associated with the MH-TCNN. For example, using a convolutional neural network with a single convolution layer or using other modeling techniques, such as baseline or polynomial fit models. For example, for a given sample set of training data, baseline models were found to have an MSE of 260.3 and the polynomial fit model was found to have an MSE of 1181.8. For the same set of training data, a one-dimensional (e.g., one-head convolutional neural network) was found to have an MSE of 191.9.

In contrast, the MH-TCNN has an MSE of 20 for the same data set. This low MSE value results in more accurate blood glucose value prediction. The use of the multi-head model with each individual variable receiving its own “head,” or convolution layer, results in a much more accurately trained model for predicting a blood glucose value for the user. This is because each variable is learned separate from other variables in individual convolution layers, and then the resulting learned convolution layers are combined at a concatenated convolution layer, which in turn undergoes learning from each of the convolution layers.

This resulting concatenated convolution layer can then output a prediction for a blood glucose level for the user.

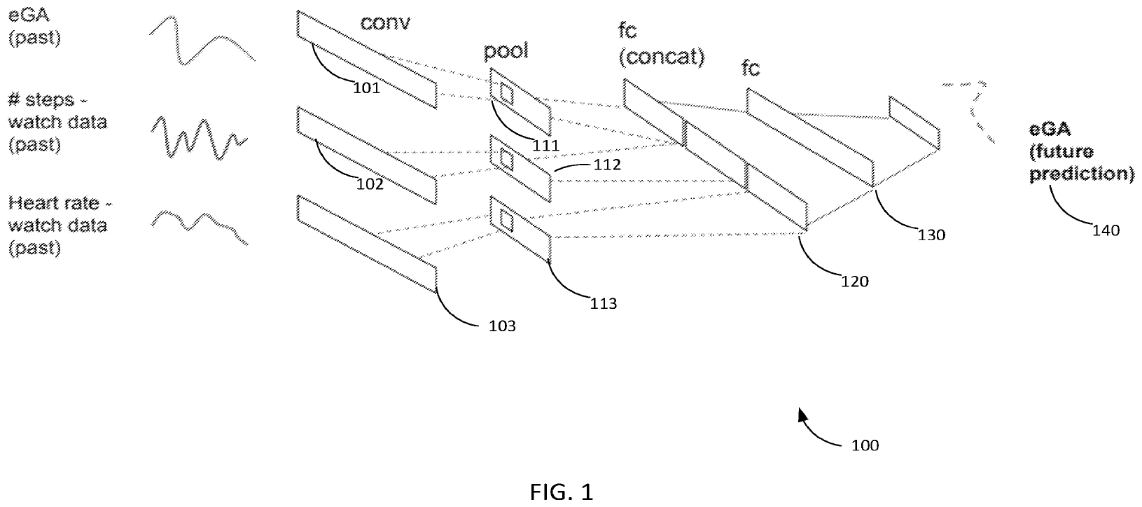

FIG. 1 illustrates a MH-TCNN (hereinafter referred to as “network”) 100 for predicting an estimated glucose average according to one aspect of the present disclosure.

The network 100 can include one or more convolution layers. For example, in an embodiment, the network 100 can include a first convolution layer 101, a second convolution layer 102, and a third convolution layer 103. In alternative embodiments, the network 100 can include more or less convolution layers.

In a further embodiment, each convolution layer of the network 100 can be associated with a single data input (e.g., a particular parameter, measurement, or other value). Each convolutional layer receives input data and abstracts the input data to a feature map through the process of convolution. In a further embodiment, each convolution layer of the network 100 can be associated with a specific, single type of data. For example, as illustrated in FIG. 1, first convolution layer 101 can be associated with an eGA value for a user, second convolution layer 102 can be associated with a number of steps taken by the user, and third convolution layer 103 can be associated with a heart rate of the user. Each convolution layer can then process individual data inputs to create an output feature map (or activation map) from each convolution layer. The feature map or activation map is an output of a layer after a particular filter of weights has been applied to that layer. The filter can be drawn across the layer, and each position across the filter results in an activation of a “neuron” or node in the layer. The resulting output is collected in a feature map. This process can be repeated across the entire layer, resulting in the feature mapIn a further embodiment, the output of each convolution layer can be output as the feature map with a shape such as (number of inputs)×(feature map height)×(feature map width)×(feature map channels) or other suitable output shapes. The convolution layers are split because each input can have different temporal correlation principles, but each input has some latent information that affects the eGA value.

The outputs of one or more convolution layers can be received by one or more pooling layers, such as first pooling layer 111, second pooling layer 112, and third pooling layer 113. Pooling layers can process portions of the output feature map from one or more convolutional layers by, for example, selecting a filter and sliding it over the output feature map and performing a pooling operation, which results in an output from the pooling layer based on the currently selected values from the output feature map(s) (e.g., currently under the filter). Examples of pooling operations can include max pooling and average pooling.

Outputs from the pooling layers can then be provided to a concatenation layer 120. The concatenation layer 120 receives the outputs from the pooling layers and concatenates the outputs along a particular dimension. For example, the concatenation layer 120 can receive a plurality of outputs from a plurality of pooling layers and combine those inputs along a specified dimension that each of the plurality of outputs has in common with the other outputs.

The concatenation layer 120 can be used as an input for a final convolution layer 130. Like the above-described convolution layers, the final convolutional layer 130 receives input data and abstracts the input data to a feature map through the process of convolution. The output of the final convolution layer 130 is an estimated glucose average (“eGA”) 140.

The network 100 can be trained using general training data (e.g., a pre-defined training data set encompassing a variety of users and data values) and/or trained to a particular user over time. For example, the network 100 can be trained using a general data set and then reinforced over time from specific patterns associated with the user.

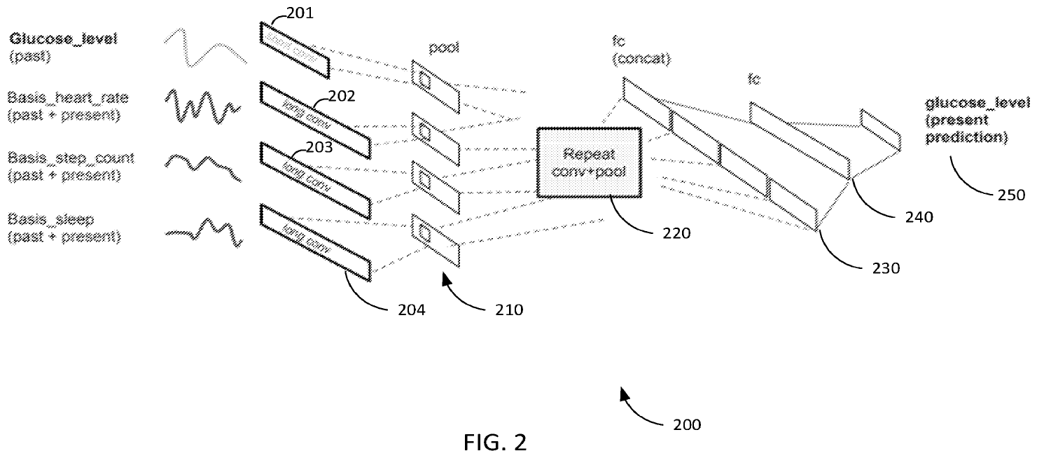

In a further embodiment, different convolution layers can have different input layer sizes. For example, FIG. 2 illustrates a multi-head convolutional neural network (“network”) 200 for predicting an estimated glucose average according to one aspect of the present disclosure. Similar to the network 100 described above with regards to FIG. 1, the network 200 can include two or more convolutional layers, such as convolution layers 201-204 and one or more pooling layers 210. However, as shown, different convolutional layers can have different input layer, or head, sizes. For example, the input head size for convolution layer 201 may be shorter than an input head size for convolution layer 202 because the input for convolution layer 201 is only a blood glucose level (e.g., an average) from the past hour, while the input for the convolution layer 202 may be an average heart rate from the past hour and a present heart rate, a heart rate from the past six hours, or any other variation in time duration.

In another example, a non-temporal input data can be assigned to a larger input size in order to align the non-temporal data with temporal data. For example, heart rate data can be continuously collected over time and will have different values at different times. However, sleep data is a binary value that is not temporal; either the user is asleep or awake. In order to align this non-temporal sleep data with the temporal data, such as heart rate, times when the user is awake can be assigned to a value of 0 and times when the user is asleep can be assigned to a value of 1. This allows non-temporal sleep data to be represented temporally as a sequence of Os and Is illustrating when the user goes to sleep and when the user wakes up. This would require a much longer input duration than, for example, the average blood glucose level over the past hour in convolution layer 201. Therefore, a convolution layer such as convolution layer 204 can have a much larger input size than the input for convolution layer 201 (e.g., a larger number of input data points for the convolution layer 204).

Additionally, the network 200 illustrated in FIG. 2 shows an example of performing various stages of convolution and pooling (see repeat convolution and pooling step 220). In a further embodiment, the network 200 can include additional convolution layers and/or pooling layers that can perform convolution and/or pooling on a subset of outputs of pooling layers 210. This repeat convolution and pooling step 220 can be performed any number of times. Then, as described with regards to network 100 in FIG. 1, outputs of pooling layers can be concatenated into a concatenation layer 230, which is then used as an input into a final convolution layer 240. The output of this final convolution layer 250 is an estimated blood glucose value 250.

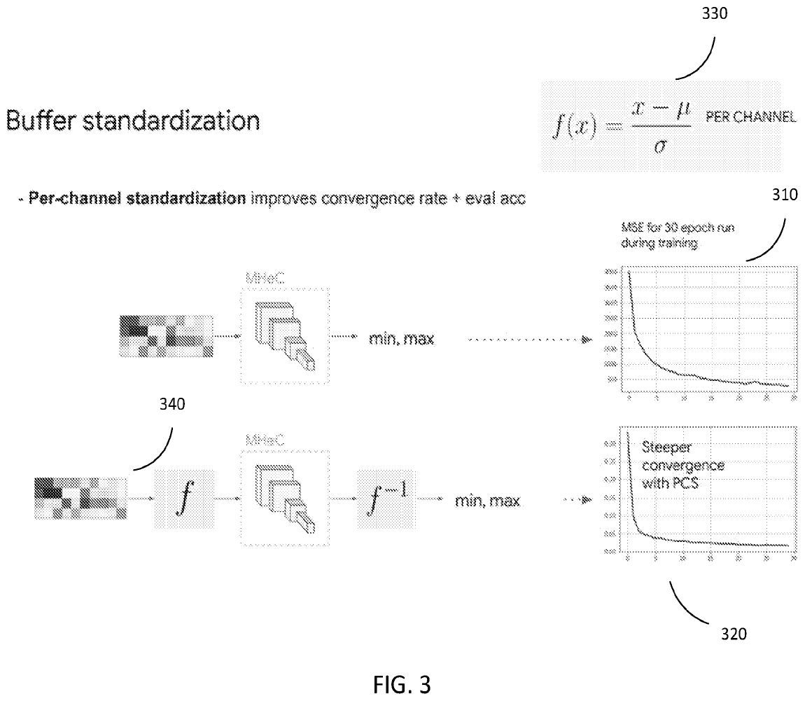

FIG. 3 illustrates buffer standardization for convolution layers in a multi-head convolutional neural network according to one aspect of the present disclosure. Buffer standardization improves the convergence rate of the MH-TCNN during training and, therefore, improves the eventual evaluation accuracy of the MH-TCNN for estimating a blood glucose level of the user. As shown in FIG. 3, without buffer standardization (310), the MH-TCNN takes longer to converge in comparison to per-channel standardization for each head, or input convolution layer, for the MH-TCNN (320). Per-channel buffer standardization is performed by applying a function 330 to the output feature map of a convolution layer 340 to standardize a range of values for input into each convolution layer.

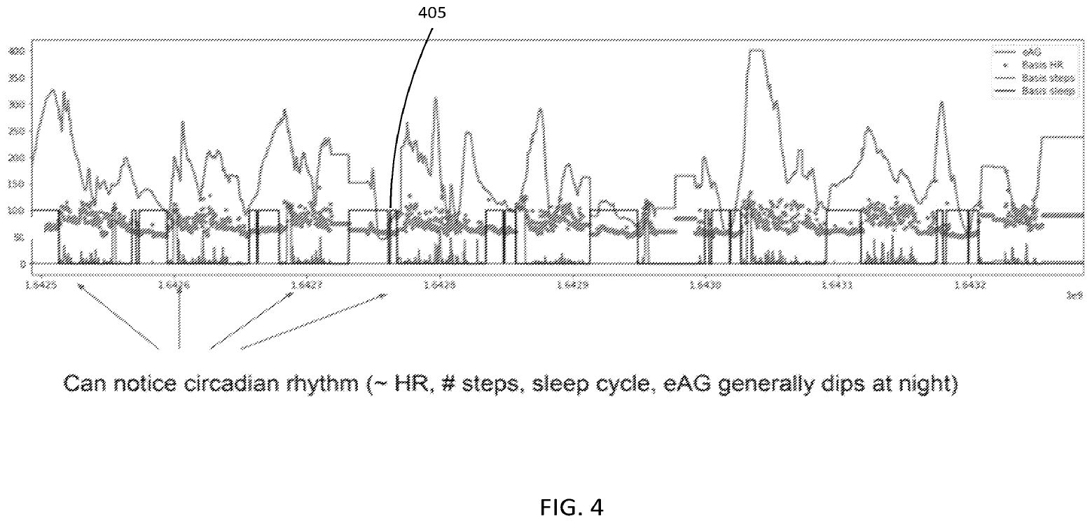

FIG. 4 illustrates lifting non-temporal data inputs for a convolutional neural network according to one aspect of the present disclosure. As noted above with regard to FIG. 2, non-temporal data such as sleep and wake times, meal times, and other instances of events that are not measured over time can be “lifted,” or assigned 0 or 1 values based on when the event occurs, in order to align the non-temporal data with temporal data, such as heart rate, average blood glucose level, steps taken, and the like. For example, in FIG. 4, sleep is tracked as a binary 0 or 1 in curve 405, where 0 indicates times when the user is awake and 1 indicates times that the user is asleep. This “lifting” therefore transforms non-temporal data (e.g., occurrence of discrete events, such as meals eaten, falling asleep and waking, and the like) into a temporal curve that transitions between the 0 and 1 values. By lifting or aligning non-temporal data with temporal data, convolution layers can receive input with similar dimensions to other convolution layers, which enables easier alignment when outputs of pooling layers are concatenated.

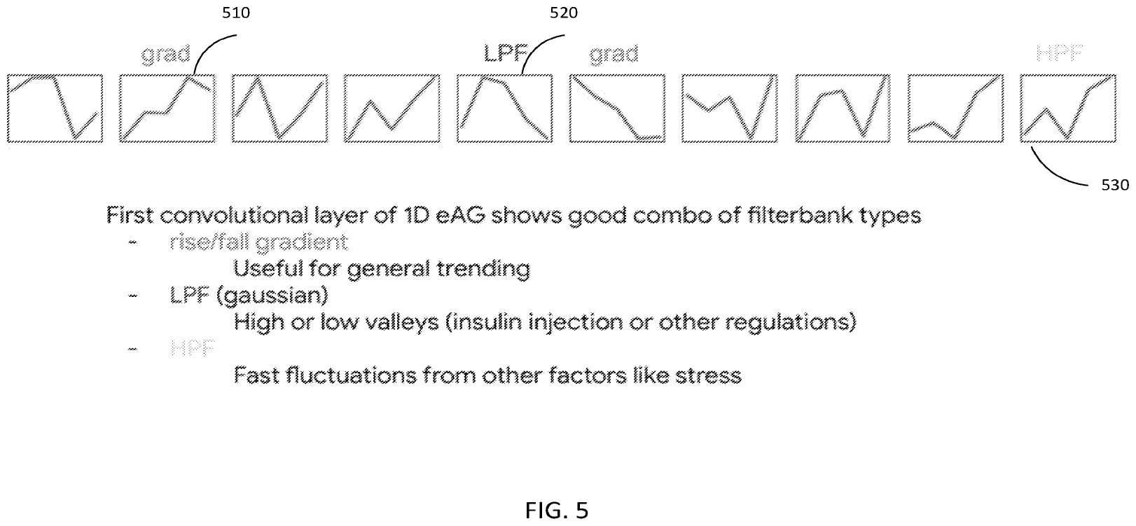

FIG. 5 illustrates patterns noticed in convolutional layers for various data inputs according to one aspect of the present disclosure. As shown, based on the type of data input into a convolution layer and a type of filter used by a pooling layer associated with the convolutional layer, different patterns can emerge, as well as indications for what types of filters are best suited to analyze the output feature maps of the convolution layers for particular data types. For example, gradient rise and fall filters can be useful for general trending data (e.g., blood glucose level, heart rate, etc.), such as in pattern 510. Low pass filters, especially Gaussian low-pass filters, can be useful for data inputs that will have high or low valleys, such as when insulin injections occur or other medicine is introduced (as shown in pattern 520). Additionally, high pass filters can lead to good outputs for data inputs with fast fluctuations, such as stress indicators, sugar consumed from meals, and the like (as shown in example 530).

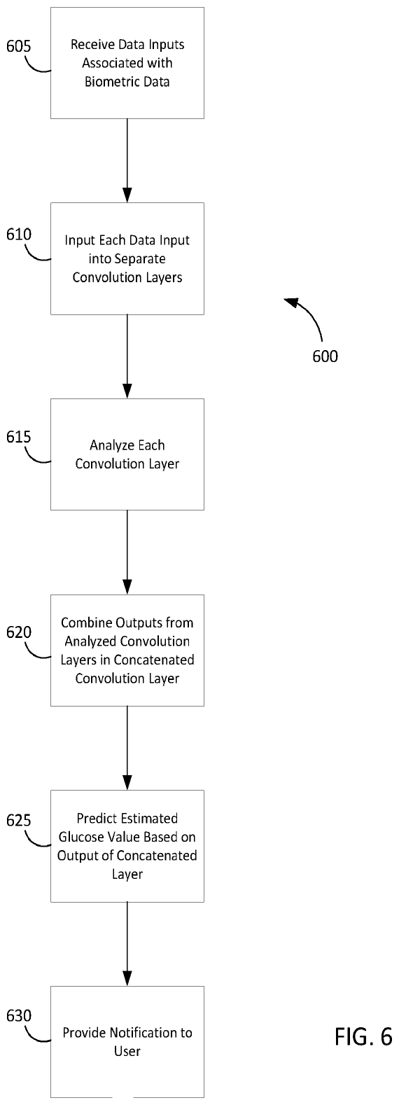

FIG. 6 illustrates a method 600 for determining a glucose value for the user (e.g., predicting an estimated glucose value) according to one aspect of the present disclosure. In a further embodiment, the method 600 can be stored as instructions in a non-transitory, computer-readable medium, such as a memory, and can be performed by one or more processors, such as one or more processors of a computing device. FIG. 6 depicts steps performed in a particular order for purposes of illustration and discussion. Those of ordinary skill in the art, using the disclosures provided herein, will understand that various steps of the method 600 or any of the other methods disclosed herein may be adapted, modified, rearranged, performed simultaneously, or modified in various ways without deviating from the scope of the present disclosure.

At block 605, the method 600 includes receiving data inputs associated with biometric data of the user and/or other data associated with the user. In a further embodiment, the received data inputs can include a past measured eGA value for a user for a period of time, such as the last twenty minutes, the last hour, the last twelve hours, and the like.

In a further embodiment, the received data input can also include basal insulin data, bolus insulin data, nutrition data (e.g., calories consumed, macronutrients consumed, and the like), sleep data (e.g., hours of sleep per day for the user, time the user fell asleep, time the user woke up, and the like), stressor data, hypoglycemic event data, illness data (type of illness experienced, duration of illness, medicines consumed, lifestyle changes such as extra sleep or hydration performed during the duration of the illness, and the like), exercise data (e.g., type and duration of exercise, varied heart rate data, calories consumed, and the like), heart rate data, air temperature data, skin temperature data, number of steps data (e.g., a number of steps the user has taken in a given time frame), and galvanic skin response data.

These data inputs can be obtained in various ways. For example, a user may be required to enter nutrition data into a software application on a computing device, such as entering nutrition data related to a meal the user has consumed. As another example, data can be collected by one or more sensors, such as sensors on a smart wearable for detecting heart beat, skin temperature, a number of steps, and the like.

Various timeframes for temporal data (e.g., heart beat, skin temperature, number of steps, stress data, and the like) can be defined by the user and/or be determined by other means, such as having a default timeframe for specific temporal data inputs, a predefined time range for all temporal data inputs, and the like.

Non-temporal data can be determined via user input (e.g., a user entering nutrition or sleep data) or by one or more sensors, such as a CGM detecting a hypoglycemic event, a smart wearable or smartphone detecting a user exercising, a weather software application providing weather data (e.g., air temperature) as a data input, and the like.

At block 610, the method 600 includes inputting each received data input into a separate convolution layer of a multi-headed temporal convolutional neural network. As described above with regards to FIGS. 1 and 2, networks 100 and 200 include individual convolution layers for each data input in order to learn how each data input can affect the resulting eGA value.

In a further embodiment, inputting each received data input into a separate convolution layer can include providing different input layer sizes to different data input types, as described above with regards to FIG. 2. For example, input head size for a first convolution layer may be shorter than an input head size for a second convolution layer because the input for the first convolution layer is only a blood glucose level (e.g., an average) from the past hour, while the input for the second convolution layer may be an average heart rate from the past hour and a present heart rate, a heart rate from the past six hours, or any other variation in time duration.

In a further embodiment, after assigning each data input to a convolution layer, buffer standardization can be performed. Buffer standardization improves the convergence rate of the MH-TCNN during training and, therefore, improves the eventual evaluation accuracy of the MH-TCNN for estimating a blood glucose level of the user. As shown in FIG. 3, without buffer standardization, the MH-TCNN takes longer to converge in comparison to per-channel standardization for each head, or input convolution layer, for the MH-TCNN. Per-channel standardization is performed by applying a function to the output feature map of a convolution layer.

At block 615, the method 600 includes analyzing each convolution layer associated with each data input. For example, as described above with regards to FIGS. 1 and 2, each convolution layer can undergo convolution to output a feature map. The feature map can have n dimensions and is representative of certain features of the input data. In order to generate the feature map, during convolution, one or more functions can be applied to the particular data input associated with the convolution layer, such as an activation function.

In a further embodiment, after analyzing each convolution layer and receiving an output feature map from each convolution layer, the generated feature maps can be provided to pooling layers. As described above with regards to FIG. 1, pooling layers can process portions of the output feature map from one or more convolutional layers by, for example, selecting a filter and sliding it over the output feature map and performing a pooling operation, which results in an output from the pooling layer based on the currently selected values from the output feature map(s) (e.g., currently under the filter). Examples of pooling operations can include max pooling and average pooling.

In a further embodiment, outputs from the pooling layers can then be provided to a concatenation layer. The concatenation layer receives the outputs from the pooling layers and concatenates the outputs along a particular dimension. For example, the concatenation layer can receive a plurality of outputs from a plurality of pooling layers and combine those inputs along a specified dimension that each of the plurality of outputs has in common with the other outputs.

At block 620, the method 600 includes combining the outputs of each respective convolution layers (and/or pooling layers) into a concatenation layer. The concatenation layer can be used as an input for a final convolution layer. Like the above-described convolution layers, the final convolutional layer receives input data and abstracts the input data to a feature map through the process of convolution.

Based on the output of the final convolution layer (e.g., a glucose value), a prediction of an eGA is determined (at block 625). This eGA can be indicative of a likely blood glucose average of the blood of the user. This is especially valuable if a user is either currently not using a CGM or is experiencing a malfunction with a CGM.

In a further embodiment, the predicted eGA can include a range of possible eGA values. For example, the predicted eGA values can include an upper limit of a possible eGA value and a lower limit of a possible eGA value. This range can indicate that, while an exact estimate is not available, the possible values of the eGA of the user's blood is within these known limits. This can allow a user to take action based on this known range.

At block 630, the method 600 includes providing a notification to the user based at least in part on the predicted eGA. For example, the notification can include a first notification indicative of the user being hyperglycemic when the blood glucose value for the user is higher than a first threshold value. In another example, the notification can include a second notification indicative of the user being hypoglycemic when the blood glucose value for the user is lower than a second threshold value that is lower than the first threshold value. In a further example, the notification can include a third notification indicative of the user being at risk for becoming hypoglycemic when the blood glucose value for the user is not higher than the second threshold value by a threshold amount.

The notification can also include actionable items for a user. For example, the notification can include an indication to stop exercising if the user is exercising, to consume food or a beverage in order to avoid risks of hypoglycemia, to wake up the user, to indicate to the user that a CGM associated with the user is not working, to indicate to the user to seek medical attention, and the like. In another example, the notification can simply include outputting the blood glucose value determined to the user for the user's observation.

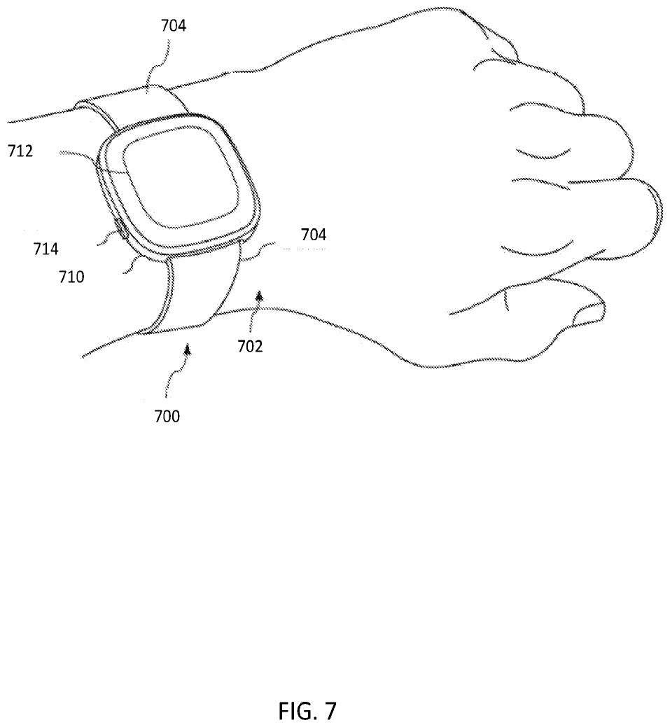

FIG. 7 depict a wearable computing device 700 according to some implementations of the present disclosure. As shown, the wearable computing device 700 can be worn, for instance, on an arm 702 (e.g., wrist) of a user. For instance, the wearable computing device 700 can include a band 704 and a housing 710. In some implementations, the housing 710 can include a conductive material (e.g., metal). In alternative implementations, the housing 110 can include a non-conductive material (e.g., a plastic material, a ceramic material).

The housing 710 can be coupled to the band 704. In this manner, the band 704 can be fastened to the arm 702 of the user to secure the housing 710 to the arm 702 of the user.

In some implementations, the wearable computing device 700 can include a display screen 712. The display screen 712 can display content (e.g., time, date, biometrics, etc.) for viewing by the user. In some implementations, the display screen 712 can include an interactive display screen (e.g., touchscreen or touch-free screen). In such implementations, the user can interact with the wearable computing device 700 via the display screen 712 to control operation of the wearable computing device 700.

In some implementations, the wearable computing device 700 can include one or more input devices 714 that can be manipulated (e.g., pressed) by the user to interact with the wearable computing device 700. For instance, the one or more input devices 714 can include a mechanical button that can be manipulated (e.g., pressed) to interact with the wearable computing device 700. In some implementations, the one or more input devices 714 can be manipulated to control operation of a backlight (not shown) associated with the display screen 712. It should be understood that the one or more input device 714 can be configured to allow the user to interact with the wearable computing device 600 in any suitable manner. For instance, in some implementations, the one or more input devices 714 can be manipulated by the user to navigate through content (e.g., one or more menu screens) displayed on the display screen 712.

While wearable computing device 700 is illustrated as an example computing device, it can be contemplated that details of the present disclosure can be implemented on other types of computing devices, such as smart cellular telephones, personal computers, tablet computers, personal digital assistants, laptop computers, and the like.

In a further embodiment, the wearable computing device 700 can include one or more sensors for collecting biometric data related to the user. For example, the wearable computing device 700 can include a heart rate sensor, an air temperature sensor, a skin temperature sensor, and other sensors for collecting data for use in the network 100 or network 200.

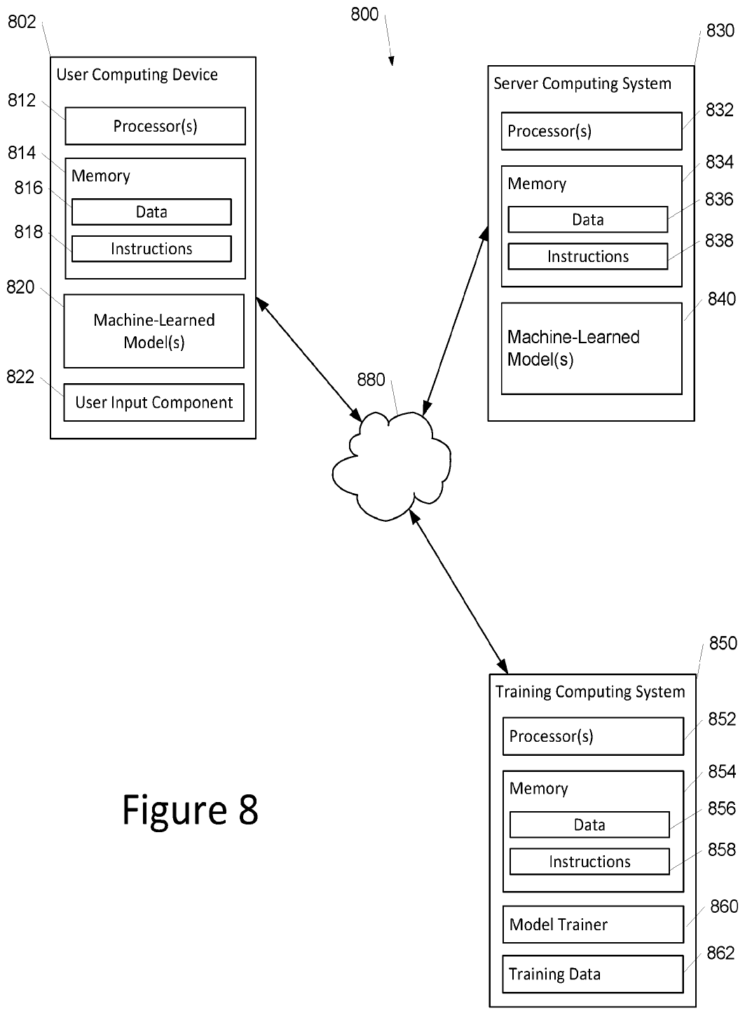

FIG. 8 depicts a block diagram of an example computing system 800 that performs eGA prediction according to example embodiments of the present disclosure. The system 800 includes a user computing device 802, a server computing system 830, and a training computing system 850 that are communicatively coupled over a network 880.

The user computing device 802 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 802 includes one or more processors 812 and a memory 814. The one or more processors 812 can be any suitable processing device (e.g., a processor core, a microprocessor, an ASIC, an FPGA, a controller, a microcontroller, etc.) and can be one processor or a plurality of processors that are operatively connected. The memory 814 can include one or more non-transitory computer-readable storage media, such as RAM, ROM, EEPROM, EPROM, flash memory devices, magnetic disks, etc., and combinations thereof. The memory 814 can store data 816 and instructions 818 which are executed by the processor 812 to cause the user computing device 802 to perform operations.

In some implementations, the user computing device 802 can store or include one or more OVERALL models 820. For example, the OVERALL models 820 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Some example machine-learned models can leverage an attention mechanism such as self-attention. For example, some example machine-learned models can include multi-headed self-attention models (e.g., transformer models). Example L models 820 are discussed with reference to FIGS. 1 and 2.

In some implementations, the one or more models 820 can be received from the server computing system 830 over network 880, stored in the user computing device memory 814, and then used or otherwise implemented by the one or more processors 812. In some implementations, the user computing device 802 can implement multiple parallel instances of a single model 820 (e.g., to perform parallel eGA prediction across multiple instances of eGA-related data).

More particularly, the models 820 can include one or more convolution layers. For example, models 820 can include a first convolution layer, a second convolution layer, and a third convolution layer. It is to be understood that there can be more or less convolution layers in the network.

In a further embodiment, each convolution layer of the models 820 can be associated with a single data input (e.g., a particular parameter, measurement, or other value). Each convolutional layer receives input data and abstracts the input data to a feature map through the process of convolution. In a further embodiment, each convolution layer of the models 820 can be associated with a specific, single type of data. For example, as illustrated in FIG. 1, a first convolution layer can be associated with an eGA value for a user, a second convolution layer can be associated with a number of steps taken by the user, and a third convolution layer can be associated with a heart rate of the user. Each convolution layer can then process individual data inputs to create an output from each convolution layer. In a further embodiment, the output of each convolution layer can be output as the feature map with a shape such as (number of inputs) x (feature map height) x (feature map width) x (feature map channels) or other suitable output shapes. The convolution layers are split because each input can have different temporal correlation principles, but each input has some latent information that affects the eGA value.

The outputs of one or more convolution layers can be received by one or more pooling layers, such as a first pooling layer, a second pooling layer, and a third pooling layer. Pooling layers can process portions of the output feature map from one or more convolutional layers by, for example, selecting a filter and sliding it over the output feature map and performing a pooling operation, which results in an output from the pooling layer based on the currently selected values from the output feature map(s) (e.g., currently under the filter). Examples of pooling operations can include max pooling and average pooling.

Outputs from the pooling layers can then be provided to a concatenation layer. The concatenation layer receives the outputs from the pooling layers and concatenates the outputs along a particular dimension. For example, the concatenation layer can receive a plurality of outputs from a plurality of pooling layers and combine those inputs along a specified dimension that each of the plurality of outputs has in common with the other outputs.

The concatenation layer can be used as an input for a final convolution layer. Like the above-described convolution layers, the final convolutional layer receives input data and abstracts the input data to a feature map through the process of convolution. The output of the final convolution layer is an estimated glucose average (“eGA”).

Additionally or alternatively, one or more models 840 can be included in or otherwise stored and implemented by the server computing system 830 that communicates with the user computing device 802 according to a client-server relationship. For example, the models 840 can be implemented by the server computing system 840 as a portion of a web service (e.g., an eGA prediction service). Thus, one or more models 820 can be stored and implemented at the user computing device 802 and/or one or more models 840 can be stored and implemented at the server computing system 830.

The user computing device 802 can also include one or more user input components 822 that receives user input. For example, the user input component 822 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 830 includes one or more processors 832 and a memory 834. The one or more processors 832 can be any suitable processing device (e.g., a processor core, a microprocessor, an ASIC, an FPGA, a controller, a microcontroller, etc.) and can be one processor or a plurality of processors that are operatively connected. The memory 834 can include one or more non-transitory computer-readable storage media, such as RAM, ROM, EEPROM, EPROM, flash memory devices, magnetic disks, etc., and combinations thereof. The memory 834 can store data 836 and instructions 838 which are executed by the processor 832 to cause the server computing system 830 to perform operations.

In some implementations, the server computing system 830 includes or is otherwise implemented by one or more server computing devices. In instances in which the server computing system 830 includes plural server computing devices, such server computing devices can operate according to sequential computing architectures, parallel computing architectures, or some combination thereof.

As described above, the server computing system 830 can store or otherwise include one or more models 840. For example, the models 840 can be or can otherwise include various machine-learned models. Example machine-learned models include neural networks or other multi-layer non-linear models. Example neural networks include feed forward neural networks, deep neural networks, recurrent neural networks, and convolutional neural networks. Some example machine-learned models can leverage an attention mechanism such as self-attention. For example, some example machine-learned models can include multi-headed self-attention models (e.g., transformer models). Example models 840 are discussed with reference to FIGS. 1 and 2.

The user computing device 802 and/or the server computing system 830 can train the models 820 and/or 840 via interaction with the training computing system 850 that is communicatively coupled over the network 880. The training computing system 850 can be separate from the server computing system 830 or can be a portion of the server computing system 830.

The training computing system 850 includes one or more processors 852 and a memory 854. The one or more processors 852 can be any suitable processing device (e.g., a processor core, a microprocessor, an ASIC, an FPGA, a controller, a microcontroller, etc.) and can be one processor or a plurality of processors that are operatively connected. The memory 854 can include one or more non-transitory computer-readable storage media, such as RAM, ROM, EEPROM, EPROM, flash memory devices, magnetic disks, etc., and combinations thereof. The memory 854 can store data 856 and instructions 858 which are executed by the processor 852 to cause the training computing system 850 to perform operations. In some implementations, the training computing system 850 includes or is otherwise implemented by one or more server computing devices.

The training computing system 850 can include a model trainer 860 that trains the machine-learned models 820 and/or 840 stored at the user computing device 802 and/or the server computing system 830 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 860 can perform a number of generalization techniques (e.g., weight decays, dropouts, etc.) to improve the generalization capability of the models being trained.

In particular, the model trainer 860 can train the OVERALL models 820 and/or 840 based on a set of training data 862. The training data 862 can include, for example, generic training data gathered from a variety of users (e.g., typical eGA, typical number of steps taken per hour, typical heart rate, and the like for each user). The training data 862 can also include user-specific training data (e.g., data from a CGM of the user indicating averaged eGA for the particular user, etc.).

In some implementations, if the user has provided consent, the training examples can be provided by the user computing device 802. Thus, in such implementations, the model 820 provided to the user computing device 802 can be trained by the training computing system 850 on user-specific data received from the user computing device 802. In some instances, this process can be referred to as personalizing the model.

The model trainer 860 includes computer logic utilized to provide desired functionality. The model trainer 860 can be implemented in hardware, firmware, and/or software controlling a general purpose processor. For example, in some implementations, the model trainer 860 includes program files stored on a storage device, loaded into a memory and executed by one or more processors. In other implementations, the model trainer 860 includes one or more sets of computer-executable instructions that are stored in a tangible computer-readable storage medium such as RAM, hard disk, or optical or magnetic media.

The network 880 can be any type of communications network, such as a local area network (e.g., intranet), wide area network (e.g., Internet), or some combination thereof and can include any number of wired or wireless links. In general, communication over the network 880 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The machine-learned models described in this specification may be used in a variety of tasks, applications, and/or use cases.

In some implementations, the input to the machine-learned model(s) of the present disclosure can be text or natural language data. The machine-learned model(s) can process the text or natural language data to generate an output. As an example, the machine-learned model(s) can process the natural language data to generate a language encoding output. As another example, the machine-learned model(s) can process the text or natural language data to generate a latent text embedding output. As another example, the machine-learned model(s) can process the text or natural language data to generate a translation output. As another example, the machine-learned model(s) can process the text or natural language data to generate a classification output. As another example, the machine-learned model(s) can process the text or natural language data to generate a textual segmentation output. As another example, the machine-learned model(s) can process the text or natural language data to generate a semantic intent output. As another example, the machine-learned model(s) can process the text or natural language data to generate an upscaled text or natural language output (e.g., text or natural language data that is higher quality than the input text or natural language, etc.). As another example, the machine-learned model(s) can process the text or natural language data to generate a prediction output.

In some implementations, the input to the machine-learned model(s) of the present disclosure can be statistical data. Statistical data can be, represent, or otherwise include data computed and/or calculated from some other data source. The machine-learned model(s) can process the statistical data to generate an output. As an example, the machine-learned model(s) can process the statistical data to generate a recognition output. As another example, the machine-learned model(s) can process the statistical data to generate a prediction output. As another example, the machine-learned model(s) can process the statistical data to generate a classification output. As another example, the machine-learned model(s) can process the statistical data to generate a segmentation output. As another example, the machine-learned model(s) can process the statistical data to generate a visualization output. As another example, the machine-learned model(s) can process the statistical data to generate a diagnostic output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output. As another example, the machine-learned model(s) can process the sensor data to generate a detection output.

In some cases, the machine-learned model(s) can be configured to perform a task that includes encoding input data for reliable and/or efficient transmission or storage (and/or corresponding decoding). For example, the task may be an audio compression task. The input may include audio data and the output may comprise compressed audio data. In another example, the input includes visual data (e.g. one or more images or videos), the output comprises compressed visual data, and the task is a visual data compression task. In another example, the task may comprise generating an embedding for input data (e.g. input audio or visual data).

FIG. 8 illustrates one example computing system that can be used to implement the present disclosure. Other computing systems can be used as well. For example, in some implementations, the user computing device 802 can include the model trainer 860 and the training dataset 862. In such implementations, the models 820 can be both trained and used locally at the user computing device 802. In some of such implementations, the user computing device 802 can implement the model trainer 860 to personalize the models 820 based on user-specific data.

Further to the descriptions above, a user may be provided with controls allowing the user to make an election as to both if and when systems, programs, or features described herein may enable collection of user information (e.g., information about a user's social network, social actions, or activities, profession, a user's preferences, or a user's current location), and if the user is sent content or communications from a server. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user. To that end, any information collected as described herein relating to the user will be kept private and confidential and will not be improperly used or published.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. A method for determining a glucose value for a user, the method comprising:
receiving a plurality of data inputs associated with biometric data of the user, the plurality of data inputs including at least one data input representative of a past estimated glucose value of the user;
processing the plurality of data inputs with a multi-headed temporal convolutional neural network to generate a blood glucose value for the user; and
providing a notification to the user based at least in part on the blood glucose value.

2. The method of claim 1, wherein processing the plurality of data inputs with a multi-headed temporal convolutional neural network to generate a blood glucose value for the user comprises:
inputting each data input of the plurality of data inputs into a corresponding convolution layer of the multi-headed temporal convolutional neural network;
analyzing each individual convolution layer for each of the data inputs separately from other individual convolution layers;
combining outputs from the analyzed individual convolution layers into a concatenated convolution layer; and
generating the blood glucose value for the user based on an output of the concatenated convolution layer.

3. The method of claim 1, wherein the plurality of data inputs includes at least one data input representative of a data input selected from the group of data inputs consisting of basal insulin data, bolus insulin data, nutrition data, sleep data, stressor data, hypoglycemic event data, illness data, exercise data, heart rate data, air temperature data, skin temperature data, number of steps data, and galvanic skin response data.

4. The method of claim 1, wherein analyzing each individual convolution layer includes performing per-layer standardization for each convolution layer.

5. The method of claim 1, the method further comprising performing lifting on non-temporal data inputs of the plurality of data inputs to align the non-temporal data inputs with temporal data inputs of the plurality of data inputs.

6. The method of claim 1, wherein at least one individual convolution layer of the multi-headed temporal convolutional neural network has an input layer size different than other individual convolution layers of the multi-headed temporal convolutional neural network.

7. The method of claim 1, wherein the blood glucose value includes a range of blood glucose values.

8. The method of claim 1, wherein:
the notification includes a first notification indicative of the user being hyperglycemic when the blood glucose value for the user is higher than a first threshold value;
the notification includes a second notification indicative of the user being hypoglycemic when the blood glucose value for the user is lower than a second threshold value that is lower than the first threshold value; and
the notification includes a third notification indicative of the user being at risk for becoming hypoglycemic when the blood glucose value for the user is not higher than the second threshold value by a threshold amount.

9. The method of claim 8, wherein the notification includes a fourth notification instructing the user to stop performing exercise or to consume food.

10. A computing device, the computing device comprising:
one or more processors; and
a non-transitory, computer-readable medium comprising instructions that, when executed by the one or more processors, cause the one or more processors to perform a process, the process comprising:
receiving a plurality of data inputs associated with biometric data of a user, the plurality of data inputs including at least one data input representative of a past estimated glucose value of the user;
processing the plurality of data inputs with a multi-headed temporal convolutional neural network to generate a blood glucose value for the user; and
providing a notification to the user based at least in part on the blood glucose value.

11. The computing device of claim 10, wherein processing the plurality of data inputs with a multi-headed temporal convolutional neural network to generate a blood glucose value for the user comprises:
inputting each data input of the plurality of data inputs into a corresponding convolution layer of the multi-headed temporal convolutional neural network;
analyzing each individual convolution layer for each of the data inputs separately from other individual convolution layers;
combining outputs from the analyzed individual convolution layers into a concatenated convolution layer; and
generating the blood glucose value for the user based on an output of the concatenated convolution layer.

12. The computing device of claim 10, the process further comprising performing lifting on non-temporal data inputs of the plurality of data inputs to align the non-temporal data inputs with temporal data inputs of the plurality of data inputs.

13. The computing device of claim 10, wherein the blood glucose value includes a range of blood glucose values.

14. The computing device of claim 10, wherein:
the notification includes a first notification indicative of the user being hyperglycemic when the blood glucose value for the user is higher than a first threshold value;
the notification includes a second notification indicative of the user being hypoglycemic when the blood glucose value for the user is lower than a second threshold value that is lower than the first threshold value; and
the notification includes a third notification indicative of the user being at risk for becoming hypoglycemic when the blood glucose value for the user is not higher than the second threshold value by a threshold amount.

15. A non-transitory, computer-readable medium comprising instructions that, when executed by one or more processors, cause the one or more processors to perform a process, the process comprising:
receiving a plurality of data inputs associated with biometric data of a user, the plurality of data inputs including at least one data input representative of a past estimated glucose value of the user;
processing the plurality of data inputs with a multi-headed temporal convolutional neural network to generate a blood glucose value for the user; and
providing a notification to the user based at least in part on the blood glucose value.

16. The non-transitory, computer-readable medium of claim 15, wherein processing the plurality of data inputs with a multi-headed temporal convolutional neural network to generate a blood glucose value for the user comprises:
inputting each data input of the plurality of data inputs into a corresponding convolution layer of the multi-headed temporal convolutional neural network;
analyzing each individual convolution layer for each of the data inputs separately from other individual convolution layers;
combining outputs from the analyzed individual convolution layers into a concatenated convolution layer; and
generating the blood glucose value for the user based on an output of the concatenated convolution layer.

17. The non-transitory, computer-readable medium of claim 15, the process further comprising performing lifting on non-temporal data inputs of the plurality of data inputs to align the non-temporal data inputs with temporal data inputs of the plurality of data inputs.

18. The non-transitory, computer-readable medium of claim 15, wherein the blood glucose value includes a range of blood glucose values.

19. The non-transitory, computer-readable medium of claim 15, wherein:
the notification includes a first notification indicative of the user being hyperglycemic when the blood glucose value for the user is higher than a first threshold value;
the notification includes a second notification indicative of the user being hypoglycemic when the blood glucose value for the user is lower than a second threshold value that is lower than the first threshold value; and
the notification includes a third notification indicative of the user being at risk for becoming hypoglycemic when the blood glucose value for the user is not higher than the second threshold value by a threshold amount.

20. The non-transitory, computer-readable medium of claim 19, wherein the notification includes a fourth notification instructing the user to stop performing exercise or to consume food.
